(19) United States
(12) Patent Application Publication
(10) Pub. No.: US 2026/0232519 A1
(43) Pub. Date: Aug. 13, 2026

(54) Exoskeleton Device

(71) Applicants: The Government of the United States, as Represented by the Secretary of the Army, Fort Detrick, MD (US); The Government of the United States, as Represented by the Director of the Defense Health Agency, Fort Detrick, MD (US); The Henry M. Jackson Foundation for the Advancement of Military Medicine, Inc., Bethesda, MD (US)

(72) Inventors: Walter Lee Childers, San Antonio, TX (US); William Brett Johnson, San Antonio, TX (US); Daniel Harrison, San Antonio, TX (US); Joshua Dale Childress, San Antonio, TX (US); Ciera Annette Price, San Antiono, TX (US)

(21) Appl. No.: 19/632,514

(22) Filed: Mar. 30, 2026

Related U.S. Application Data
(63) Continuation-in-part of application No. PCT/US2024/051486, filed on Oct. 16, 2024.
(60) Provisional application No. 63/591,005, filed on Oct. 17, 2023.
(60) Provisional application No. 63/624,458, filed on Jan. 24, 2024.

(51) Int. Cl. A61H 3/00; A61F 5/01; A61H 1/02

(52) U.S. Cl. CPC A61H 3/00; A61F 5/0102; A61H 1/024; A61H 2201/0157; A61H 2201/1616; A61H 2201/163; A61H 2201/1642; A61H 2201/165

(57) ABSTRACT

An exoskeleton device includes a telescoping rod comprising at least one lower telescoping section and at least one upper telescoping section; a fracture stabilization orthosis; at least one fracture suspension mechanism connecting the lower telescoping section to the fracture stabilization orthosis; and a thigh corset connectable to the at least one upper telescoping section.

This is a Continuation-in-Part application of PCT international application PCT/US2024/051486 filed on Oct. 16, 2024, and claims priority to U.S. Ser. No. 63/591,005, filed in the US Patent and Trademark Office on Oct. 17, 2023 and U.S. Ser. No. 63/624,458 filed in the US Patent and Trademark Office on Jan. 24, 2024. The entirety of each provisional application is incorporated herein by reference in its entirety.

The present invention was made with government support from the US Army Medical Research and Development Command (USAMRDC) Combat Casualty Care Research Program (CCCRP) under Proposal Number #CO210052. The United States government has certain rights in the invention.

### FIELD OF INVENTION

The present invention is directed to an exoskeleton device. The exoskeleton device helps stabilize a limb with mobility-compromising injuries and enables an injured person to be mobile until a higher level of medical care can be obtained.

### BACKGROUND OF INVENTION

There is a need for stabilizing injured individuals having lower leg injuries (e.g., bone fractures, such as tibia and fibula bone fractures) and for enabling mobility or self-evacuation, for example, in austere or remote environments or in an environment in which medical help or evacuation is not readily available.

Prolonged care may have several deleterious impacts, in particular for emergency, medical, health care, or military personnel or units. For example, leg bone fractures require vehicle transport. Evacuation may take days during which units or teams must remain mobile. The use of a litter for an injured person may take four or more people to carry, thereby removing at least five people from essential tasks. Further, the lack of timely medical care may increase casualty rates.

Thus, there remains a need for a device that enables increased mobility for individuals with leg injuries and which allows them to ambulate independently.

### SUMMARY OF INVENTION

The invention provides in a first embodiment an exoskeleton device comprising a telescoping rod comprising at least one lower telescoping section and at least one an upper telescoping section; a fracture stabilization orthosis; at least one fracture suspension mechanism connecting the at least one lower telescoping section to the fracture stabilization orthosis; and a thigh corset connectable to the at least one upper telescoping section.

The invention provides in a second embodiment further to any of the previous embodiments an exoskeleton device in which the at least one lower telescoping section and at least one an upper telescoping section are in slidable engagement.

The invention provides in a third embodiment further to any of the previous embodiments an exoskeleton device in which the telescoping rod has an elliptical cross section.

The invention provides in a fourth embodiment further to any of the previous embodiments an exoskeleton device further comprising a knee joint connectable to the at least one lower telescoping section and at least one an upper telescoping section, for example, by one or more pylons.

The invention provides in a fifth embodiment further to any of the previous embodiments an exoskeleton device in which the knee joint has a double hinge structure that allows the exoskeleton device to be folded/unfolded and locked on either a right side or a left side of a user's body. The knee joint may have a spring-loaded latch mechanism to release or lock a hinge structure.

The invention provides in a sixth embodiment further to any of the previous embodiments an exoskeleton device further comprising an end effector connectable to an end of the at least one lower telescoping section to provide the device with a foot-like structure.

The invention provides in a seventh embodiment further to any of the previous embodiments an exoskeleton device in which the end effector is foldable up to and/or at least partially around the telescoping rod.

The invention provides in an eighth embodiment further to any of the previous embodiments an exoskeleton device in which the at least one fracture suspension mechanism comprises a linkage structure comprising at least one crossbar extending from the at least one lower telescoping section to the fracture stabilization orthosis. The linkage structure may comprise two to four crossbars.

The invention provides in a ninth embodiment further to any of the previous embodiments an exoskeleton device in which the at least one fracture suspension mechanism comprises a body having a cavity that is in slidable engagement with the at least one lower telescoping section. There may be two fracture suspension mechanisms, each comprising a body having a cavity that is in slidable engagement with the at least one lower telescoping section and a concave surface that is positioned against the fracture stabilization orthosis.

The invention provides in a tenth embodiment further to any of the previous embodiments an exoskeleton device in which the fracture stabilization orthosis comprises a fabric material wrappable around a lower leg and has a frustrum shape.

The invention provides in an eleventh embodiment further to any of the previous embodiments an exoskeleton device in which the fracture stabilization orthosis comprises a fabric wrap having a plurality of pockets, with a strut insertable into each pocket.

The invention provides in a twelfth embodiment further to any of the previous embodiments an exoskeleton device in which the fracture stabilization orthosis comprises two to twelve struts, each strut comprising at least one sheet of ultra-high-molecular-weight polyethylene, at least one sheet of aluminum, at least one sheet of a unidirectionally woven carbon fiber or fiberglass, or any combination thereof.

The invention provides in a thirteenth embodiment further to any of the previous embodiments an exoskeleton device in which the thigh corset comprises a fabric material wrappable around a user's thigh.

The invention provides in a fourteenth embodiment further to any of the previous embodiments an exoskeleton device in which the thigh corset comprises a plurality of pockets, each pocket for receiving one or more stays.

The invention provides in a fifteenth embodiment further to any of the previous embodiments an exoskeleton device further comprising an ischial sling connected to the at least one upper telescoping section above the thigh corset and wrappable underneath a user's pelvis.

The invention provides in a sixteenth embodiment further to any of the previous embodiments an exoskeleton device in which the ischial sling comprises a belt or strip of fabric extending or threaded through a foam tube serving as a pad.

The invention provides in a seventeenth embodiment further to any of the previous embodiments an exoskeleton device further comprising a silesian belt, wherein the ischial sling and silesian belt comprise a single strip of material.

The invention provides in an eighteenth embodiment further to any of the previous embodiments an exoskeleton device further comprising a shoulder strap.

### BRIEF DESCRIPTION OF THE DRAWINGS

FIG. 1 is a perspective view of an exoskeleton device according to an embodiment of the present invention in use.

FIG. 2 is a perspective view of an exoskeleton device according to another embodiment of the present invention.

FIG. 3 is a schematic diagram of a telescoping rod and end effector of the exoskeleton device.

FIG. 4A is a perspective view of a folded exoskeleton device with an end effector folded up. FIG. 4B is a bottom view of FIG. 4A. FIG. 4C is a perspective view of a knee joint with a double hinge structure according to one embodiment of the present invention.

FIG. 5 is a side view of the exoskeleton device folded in half.

FIG. 6 is a perspective view of a knee joint of the exoskeleton device according to an embodiment of the present invention.

FIG. 7A is a top view of a fracture suspension mechanism positioned or connected at the proximal ankle. FIG. 7B is a top view of a fracture suspension mechanism positioned or connected at the calf.

FIG. 8 is a perspective view of an open fracture stabilization orthosis.

FIG. 9 is a perspective view of the open fracture stabilization orthosis with a strut being insertable into a pocket.

FIG. 10 is a flat, open view of a thigh corset of the exoskeleton device according to an embodiment of the present invention.

FIG. 11 is a front view of a user wearing an exoskeleton device according to an embodiment of the present invention.

FIG. 12 is a rear perspective view of a user wearing an exoskeleton device according to an embodiment of the present invention.

FIG. 13 is a side view of a top part of an exoskeleton device with an open thigh corset.

### DETAILED DESCRIPTION OF INVENTION

The present invention is directed to an exoskeleton device having a structure which transfers or redirect loads away from an injured lower leg towards the thigh and/or pelvis. In particular, the exoskeleton device is useful for individuals with lower leg injuries (e.g., a tibia or fibular bone fracture) in prolonged field care scenarios or in situations where next-level medical care is not immediately available. The exoskeleton device may be used, for example, in battlefields, emergency situations, and difficult or austere environments where the injured persons may have to evacuate themselves without aid. In embodiments, the exoskeleton device may weigh about 4 to about 8 pounds (about 1.8 to about 3.6 kg).

In this detailed description, references to “one embodiment”, “an embodiment”, or “in embodiments” mean that the feature being referred to is included in at least one embodiment of the invention. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

As used herein “substantially”, “generally”, “about”, and other words of degree are relative modifiers intended to indicate permissible variation from the characteristic so modified (e.g., ±0.1%, ±0.5%, ±1.0%, ±2%, ±5%, ±10%, ±20%). It is not intended to be limited to the absolute value or characteristic which it modifies but rather possessing more of the physical or functional characteristic than its opposite, and preferably, approaching or approximating such a physical or functional characteristic.

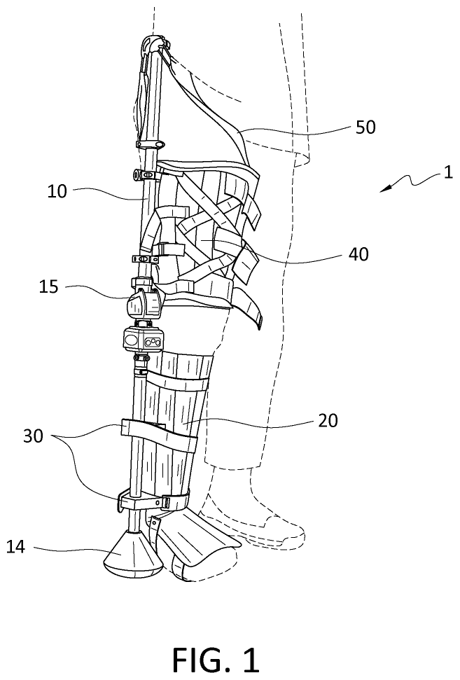

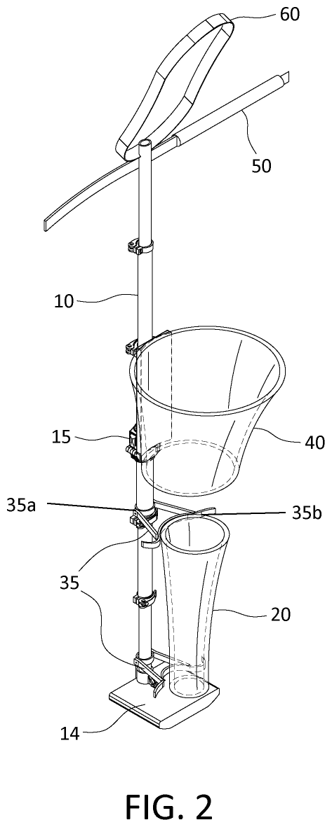

As shown in FIGS. 1-2, the exoskeleton device 1 according to the present invention comprises a telescoping rod 10; a fracture stabilization orthosis 20; at least one fracture suspension mechanism 30 connecting the telescoping rod to the fracture stabilization orthosis 20; and a thigh corset 40. The exoskeleton device 1 may also comprise an ischial sling 50.

### A. Telescoping Rod and End Effector

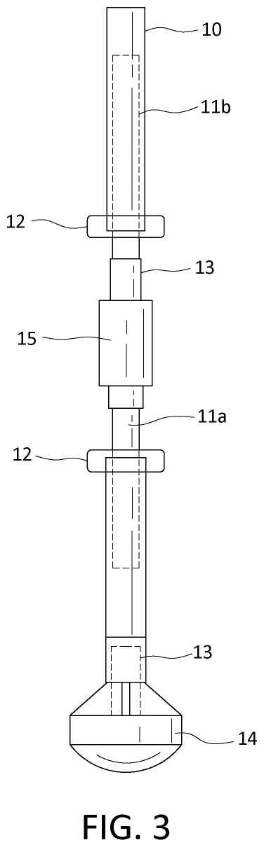

As shown in FIGS. 1-3, the telescoping rod 10 may comprise at least two telescoping sections, for example two to eight telescoping sections. In embodiments, the telescoping rod may have at least one lower telescoping section 11a (distal to the knee) and at least one upper telescoping section 11b (proximal to the knee) that may be in slidable engagement. In a specific embodiment, the telescoping rod may have four telescoping sections, that is an upper telescoping section comprising two telescoping parts that are in slidable engagement and a lower telescoping section comprising two telescoping parts that are in slidable engagement. In embodiments, the at least one lower and the at least one upper telescoping sections may be connectable to a knee joint 15 by one or more pylons.

In embodiments, the telescoping sections or parts may have a circular or elliptical cross section. An elliptical cross section may help prevent the telescoping sections and the telescoping rod from transverse plane rotation as the telescoping rod is extended and maximizes a strength to weight ratio as a bending moment in the sagittal plane is greater than in the frontal plane.

In embodiments, the telescoping rod may comprise a pin on a stationary pylon or telescoping section and a slot on a moving pylon or corresponding telescoping section to prevent rotation when extending/retracting.

The telescoping rod 10 may run along either lateral side of a user's body and can be extended or retracted to accommodate users of various sizes. In embodiments, the telescoping rod may accommodate sizes ranging from a female in the 5th percentile of height in the U.S. Army to a male in the 95th percentile of height in the U.S. Army.

The telescoping sections 11a, 11b or parts thereof may be secured using at least one securing device 12. In embodiments, the securing device may be, but is not limited to a clamp, pin, piston, or the like. The at least one lower telescoping section and the at least one upper telescoping section may allow for separate adjustability to best align the knee joint 15 and a user's anatomical knee joint.

The telescoping rod 10 may also have at least one adapter or pylon 13 connected to at least one the telescoping sections or parts thereof and/or to knee joint 15 to allow additional components to be fixed or removably attachable to the telescoping rod 10.

In specific embodiments, an end effector 14 may be connected to a lower telescoping section 11a via an adapter or pylon 13, thereby providing a foot-like structure to assist a user in walking. The end effector may have a cavity for the connection to the lower telescoping section. The foot-like structure may be configured to help a user walk in a variety of environments, such as in uneven or muddy terrain.

In embodiments, the end effector 14 may comprise a shock absorbing material, such as rubber or foam. The end effector 14 may have a rounded bottom or a rocker sole to facilitate a user's gait.

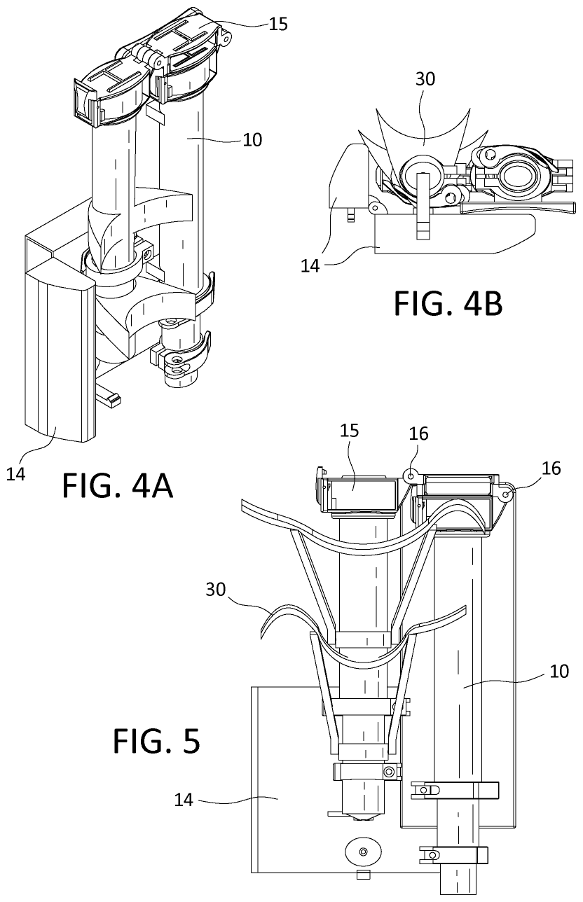

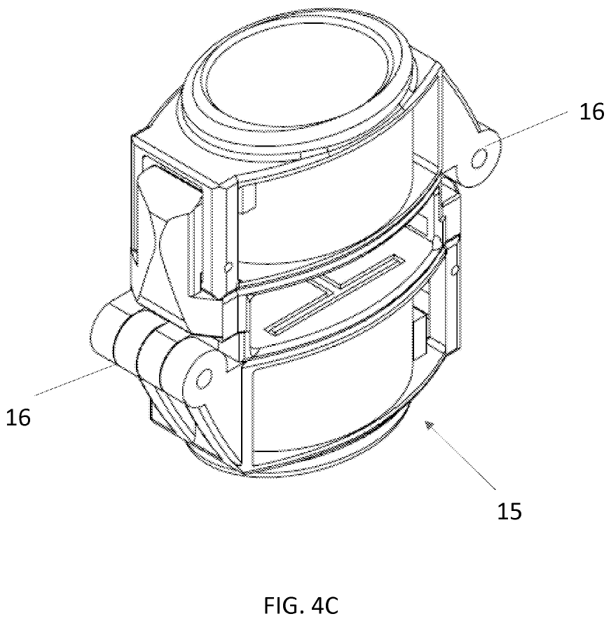

As shown in FIGS. 4A-4B, in a specific embodiment, the end effector 14 or a part thereof may be folded up to and/or at least partially around part of the telescoping rod 10 (e.g., the lower telescoping section) to reduce the amount of space required to store the exoskeleton device. The end effector may have a release mechanism or hinge that allows it to separate prior to folding.

In embodiments, the telescoping rod 10 (at least one telescoping sections 11a and 11b) may comprise a metal (for example, aluminum or titanium), plastic, or a carbon fiber laminate material, with specific fiber orientations, to optimize strength to weight ratio and manufacturability.

### B. Knee Joint

As shown in FIGS. 1-3, the telescoping rod may have a knee joint 15, for example in the middle of the telescoping rod. In embodiments, the knee joint 15 may have a single or double hinge structure 16 (FIGS. 4C-5). A double hinge structure comprising a hinge point on each side allows the exoskeleton device to be folded/unfolded and locked in both directions (e.g., up to 180 degrees) and to be folded/unfolded and locked on either the right or left side of a user's body for universal fit. In embodiments, the knee joint 15 may be foldable to allow wearers to bend their leg while sitting. The exoskeleton device may be folded, for example, in half to be compact (e.g., FIGS. 4A and 5).

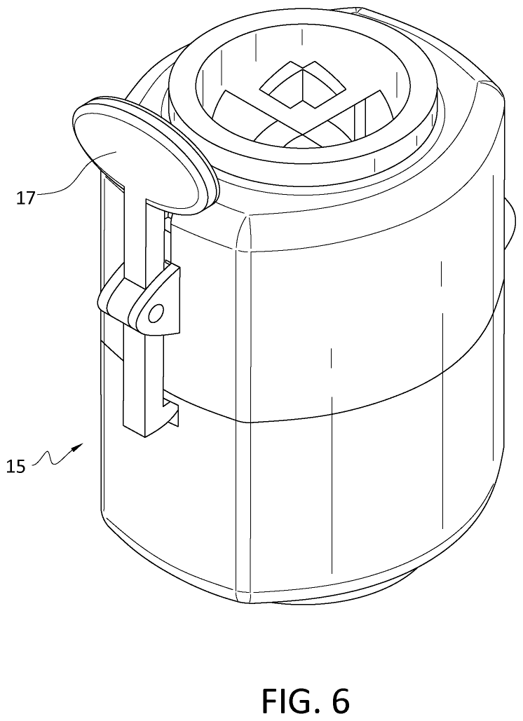

The knee joint may have a push button or spring-loaded latch mechanism 17 (FIG. 6) to release or lock the hinge structure 16. In embodiments, the knee joint may have a damper system to allow the leg to swing freely during the swing phase of gait and be stiff during the stance phase of gait. In embodiments, the knee joint may may utilize a polycentric, multi-bar system (e.g., a 4-bar knee joint).

### C. Fracture Suspension Mechanism

As shown in FIGS. 1-3, at least one fracture suspension mechanism 30 connects the telescoping rod 10, for example the at least one lower telescoping section 11a, to the fracture stabilization orthosis 20. In specific embodiments, the at least one fracture suspension mechanism 30 allows an injured lower leg to freely slide up and down the telescoping rod 10 and/or pylon as the wearer settles into the device with or during each step, which prevents that relative motion from agitating the injury or fracture. The at least one fracture suspension mechanism 30 may be removably or permanently attachable to the fracture stabilization orthosis by, in specific embodiments, a hook-and-loop fastener, belt, buckle, arms, a circumferential strap, rivets, screws, or any combination thereof. Thus, the lower leg and/or the telescoping rod may move continuously up and down.

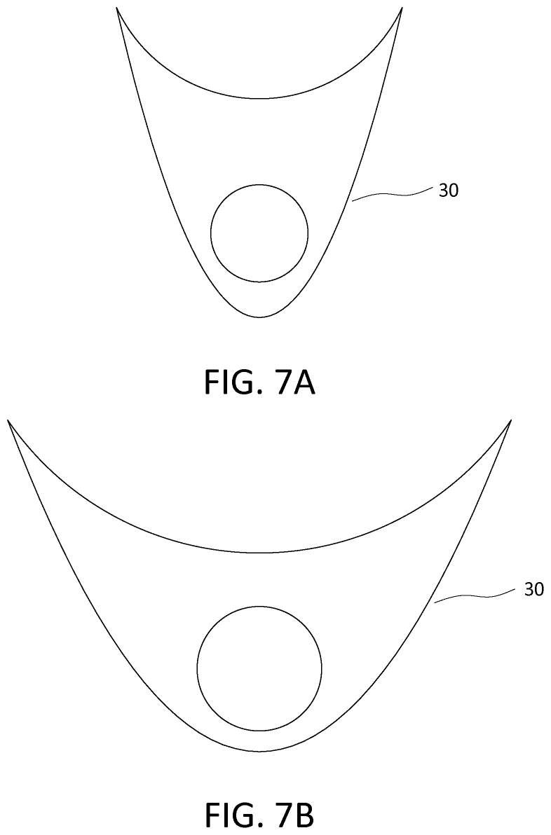

In an embodiment, the at least one fracture suspension mechanism 30 comprises a body having a cavity to retain the lower telescoping section 11a in free axial slidable engagement (FIG. 1) and a concave surface to interface with a lower limb. In a specific embodiment, there may be two fracture suspension mechanisms, one located at the proximal ankle (FIG. 7A) and another at the calf (FIG. 7B) of a user. The proximal ankle fracture suspension mechanism may have a concave surface shaped to the ankle and the calf fracture suspension mechanism may have a concave surface shaped to the surface of a calf.

This configuration may help minimize mechanical loads on a lower leg injury or fracture, while also allowing the lower leg to impart sagittal plane moments on the exoskeleton for control. The cavity may have a coating or lining to reinforce the body and/or to reduce friction between the at least fracture suspension mechanism 30 and the lower telescoping section 11a. The concave surface may broadly match a contour of the lateral lower limb for positioning against the fracture stabilization orthosis 20. In embodiments, a strap may circumferentially secure the lower limb and fracture stabilization orthosis 20 to the at least one fracture suspension mechanism 30.

In another embodiment, the at least one fracture suspension mechanism 30 comprises a linkage structure having at least one, for example two to four, crossbars 35 or other linking structures that extend from the at least one lower telescoping section 11a to the fracture stabilization orthosis 20 (FIG. 2). In embodiments, there may be one or more sets of crossbars, each set comprising two crossbars that are substantially parallel with each other. Each crossbar may have two pivot points: a first pivot point 35a on the lower telescoping section and/or on the body connected to the lower telescoping section, and a second pivot point 35b on the orthosis and/or on a surface of the crossbar abutting the orthosis. Thus, the crossbars 35 allow motion substantially in the front plane, that is along the longitudinal axis of the telescoping rod. The fracture suspension mechanism may also have a concave surface to interface with or abut the orthosis on the lower limb. The crossbars may be flexible and therefore able to accommodate lower limbs of different sizes.

The at least one crossbar may move or rotate at each pivot point and the combination of crossbar length and the relatively small angular rotation allows the fracture stabilization orthosis to move independently in the vertical direction more than the lateral direction to reduce or prevent disruption of the injury or fracture. In embodiments, having upper and lower crossbars at or near the same length ensures that the top and bottom motion of the fracture stabilization orthosis relative to the exoskeleton remains constant to prevent fracture disruption in the coronal plane. This crossbar configuration substantially reduces or prevents the axial transfer of force, while enabling the user to better control the exoskeleton in the transverse and/or sagittal plane when a user is walking, thereby reducing or preventing disruption of a bone fracture in the fracture stabilization orthosis.

In embodiments, the at least one fracture suspension mechanism may comprise at least one of foam, metal, for example aluminum or titanium, nylon, a plastic, carbon fiber laminate, or resin material (e.g., Delrin®).

### D. Fracture Stabilization Orthosis

As shown in FIGS. 1-2, the exoskeleton device comprises a fracture stabilization orthosis 20, which may be wrapped around a lower leg that has an injury or fracture (e.g., tibia or fibula bone fractures). The fracture stabilization orthosis 20 may comprise an orthosis as described in WO 2024/059018A2, which is incorporated herein by reference in its entirety.

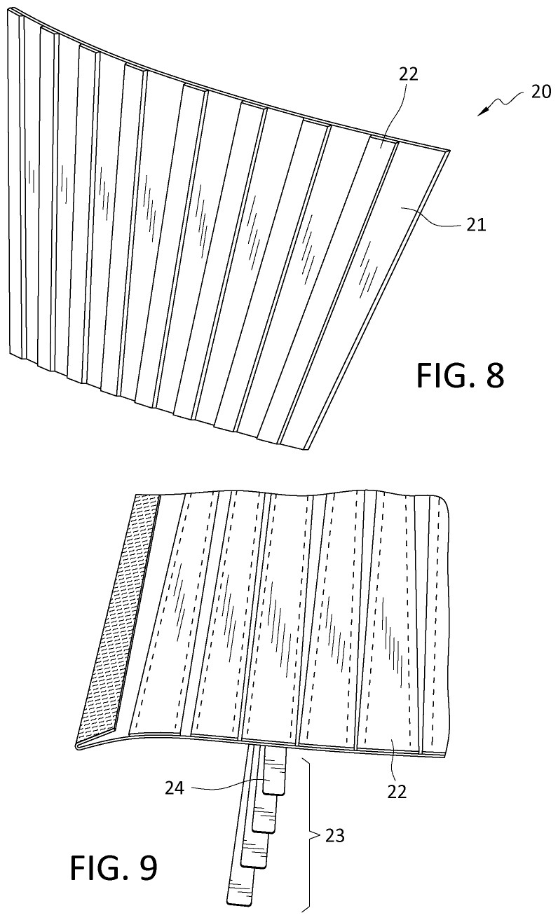

As shown in FIGS. 8-9, in embodiments, the facture stabilization orthosis 20 may comprise a fabric wrap 21, which can wrap around a user's limb to form a frustum shape. The fabric wrap may have pockets 22 for receiving and retaining a plurality of struts 23.

The plurality of struts provide stability to help immobilize a limb with a bone fracture. In embodiments, the plurality of struts may be, for example, about 4 to about 12 struts. The plurality of struts within pockets may be equally spaced along or around the fabric or fabric wrap. Each strut may be sewn into the fabric or fabric wrap or may be removably insertable into a corresponding pocket, which may substantially extend a length of the fabric or fabric wrap (e.g., a length of a lower leg).

In specific embodiments, each strut may have a width of about ⅜ inch to about ½ inch (about 0.95 cm to about 1.27 cm). The width of the pocket or sleeves may be about 1.5 cm to about 3.5 cm, for example, about 2.0 cm to about 3.0 cm. Each strut may have a length of about 40 cm to about 50 cm, for example, about 45 cm.

In specific embodiments, each strut 23 may comprise two, three, or more, for example four to eight, sheets of material 24 that can conform to the contours of the leg, while providing rigidity in other planes. The sheets may comprise, but are not limited to, aluminum, titanium, steel, nylon, carbon fiber laminate, fiberglass laminate, KEVLAR® laminate, ultra-high-molecular-weight polyethylene (UHMWPE or DYNEEMA®), carbon fiber (e.g., a unidirectionally woven carbon fiber) or a laminated composite of any combination thereof. Any material with the required degree of low stiffness and high ductility may be used that allows the fracture stabilization orthosis to accommodate the shape of the lower limb (e.g., over the fibular head or non-uniform shapes due to the injury), but then act to make the combination of the lower leg and the orthosis stiffer when the orthosis is tightened, thus stabilizing an injured or fracture site. In embodiments, the struts may be pliable so they are bendable away from a limb, for example, so that the orthosis may be fit to people with short lower legs.

In a specific embodiment, the fracture stabilization orthosis may comprise twelve struts, each strut comprising two sheets; or six struts, each strut comprising three or four sheets. In another embodiment, the fracture stabilization orthosis may comprise two to twelve struts, each strut comprising at least one sheet of ultra-high-molecular-weight polyethylene (DYNEEMA®), at least one sheet of aluminum, at least one sheet of a unidirectionally woven carbon fiber or fiberglass, or any combination thereof.

### E. Thigh Corset

As shown in FIGS. 1-2 and 10, the thigh corset 40 may comprise a fabric material or cuff that wraps around a user's thigh. The thigh corset 40 may have a plurality of pockets 42, each pocket for receiving one or more corset stays. The stays are flexible enough to conform to the shape of the thigh but also create a rigid system once the thigh corset is tightened. In embodiments, the stays may comprise carbon fiber or composite material. The thigh corset 40 may have at least one buckle 43 for securing the fabric corset 40 around a user's thigh. The fabric corset may be made of any suitable materials, including but not limited to, a breathable fabric, heavy duty canvas, nylon, and the like. In embodiments, opposing vertical ends of the thigh corset may be attached via a hook-and-loop, snap, button, or zipper fastener.

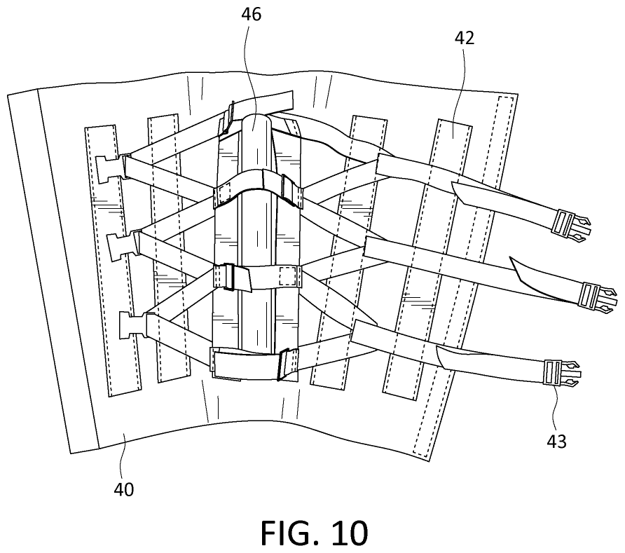

In embodiments, the thigh corset 40 attaches to the telescoping rod and/or to a pylon. In embodiments, the attachment may be by a thigh corset attachment plate or central curved junction 46 (FIG. 10) or a plate (FIG. 2). The attachment may be clipped onto or mounted to the telescoping rod and/or pylon above the knee joint. The attachment allows for permanently or removably connecting the thigh corset 40 to either side of the telescoping rod 10 (e.g., at least one upper telescoping section 11b) to fit either the right or the left leg. In embodiments, the attachment may comprise a metal (e.g., aluminum), carbon fiber material, or composite material. In a specific embodiment, the thigh corset 40 may support substantially the entire weight of a user.

### F. Ischial Sling

As shown in FIGS. 1-2 and 11, the ischial sling 50 comprises at least one strip or belt of fabric connectable to the telescoping rod 10 above the thigh corset 40 and wraps underneath a user's pelvis. In an embodiment, as shown in FIG. 2, the ischial sling 50 comprises at least one strip of fabric extending or threaded through a tube (e.g., a foam tube) serving as a pad. In embodiments, the ischial sling 50 may have a buckle, which allows the sling to be used on either side of a user's body. The fabric may be cotton, leather, nylon, canvas, or any suitable material.

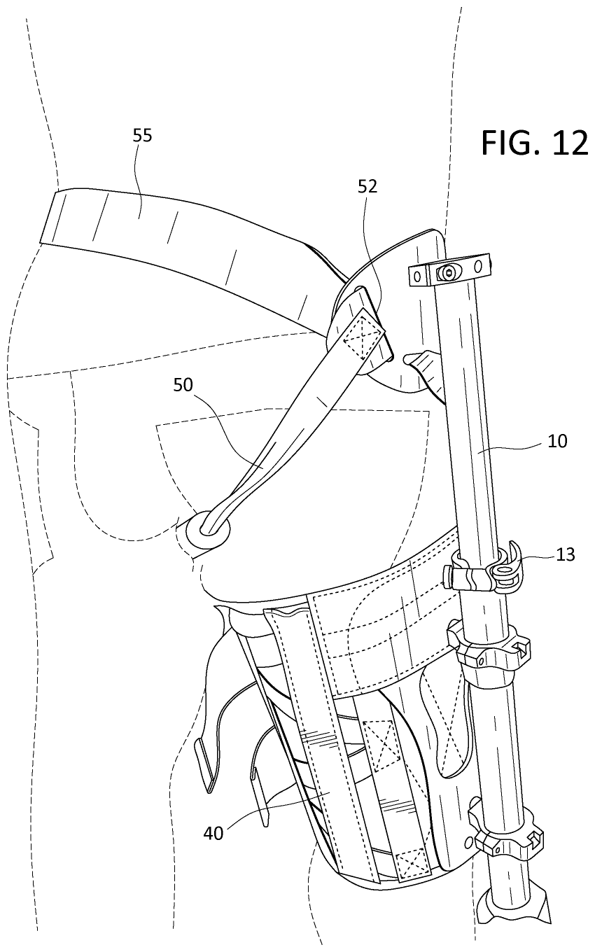

The ischial sling 50 may be connectable to the upper telescoping section 11b of the telescoping rod via a connection point or junction 52. In embodiments, as shown in FIG. 12, the connection point or junction 52 may be a carbon fiber or metal plate, which is mounted to the telescoping rod 10. In embodiments, the ischial sling 50 may have a fixed or slidable pad which allows for alignment with a user's ischial tuberosity. In one embodiment, force applied by a user's body to the telescoping rod may be distributed to the thigh corset 40 and to the ischial sling 50.

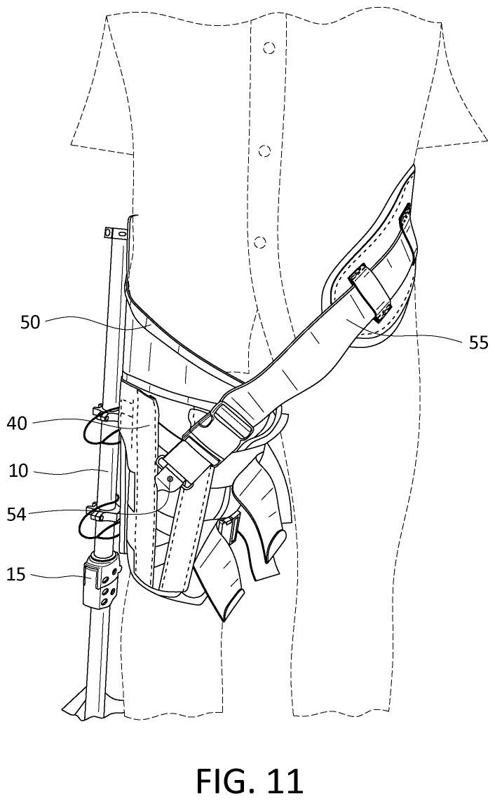

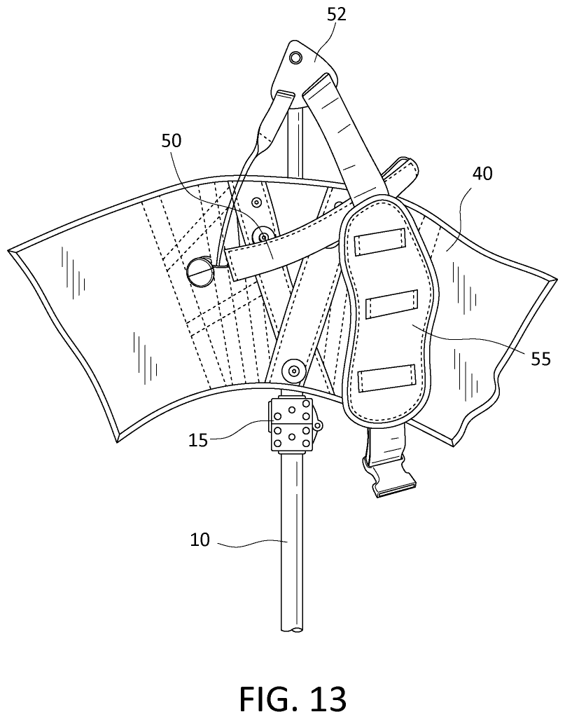

As shown in FIGS. 11-13, in a specific embodiment, exoskeleton device may further comprise a silesian belt 55. The silesian belt comprises a strip or piece of fabric with a slidable pad that overlays a user's iliac crest opposite the telescoping rod 10. One end of the silesian belt may be connectable to the front of the thigh corset via an attachment means 54 (e.g., clip, hook, buckle, pivoting loop and chafe). In embodiments, the attachment means may be a single anterior attachment means at a level of a user's ischial tuberosity.

The other end of the silesian belt may wrap across a back of a user's body through the connection point or junction 52. In specific embodiments, the ischial sling 50 and silesian belt 55 may comprise a continuous single belt or strip that is threaded through slots in the connection point or junction 52, thereby allowing tightening of the ischial sling and silesian belt together. The silesian belt 55 and/or connection point or junction 52 may be aligned with or positioned at a user's greater trochanter.

In some embodiments, ischial sling and silesian belt may be attachable to each other via a fastener including, but not limited to, a hook-and-loop fastener, zipper, buttons, or snaps.

### G. Shoulder Strap

The exoskeleton device may have a shoulder strap 60 (FIG. 2) connected to the telescoping rod 10 (e.g., at least one upper telescoping section 11b). In specific embodiment, the shoulder strap may be used with, or as an alternative to, the silesian belt.

The shoulder strap may have a buckle to allow for easy donning of the exoskeleton device. In embodiments, the shoulder strap may incorporate elastic or elastic elements to enable the user to bend their trunk while maintaining suspension. In embodiments, the shoulder strap, ischial sling, fracture stabilization orthosis, and thigh corset, or any combination thereof, may have buckles that are not interoperable and that allow for an independent ordering of connections.

### INDUSTRIAL APPLICABILITY

The present invention provides an exoskeleton device that helps stabilize a limb with movement-hindering injury and enables an injured person to be mobile until a higher level of medical care can be obtained.

Although the present invention has been described in terms of particular exemplary and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

### REFERENCE LIST

1: Exoskeleton
10: Telescoping Rod
11a: At least one Lower Telescoping Section
11b: At least one Upper Telescoping Section
12: Securing Device
13: Adapter or Pylon
14: End Effector
15: Knee Joint
16: Hinge Structure
17: Latch Mechanism
20: Fracture Stabilization Orthosis (FSO)
21: Fabric Wrap
22: Pockets
23: Struts
24: Sheets
30: At least one Fracture Suspension Mechanism
35: Crossbars
35a: Pivot Points
40: Thigh Corset
42: Corset Pockets
43: Corset Buckle
46: Thigh corset attachment plate
50: Ischial Sling
52: Connection Point or Junction
54: Attachment Means
55: Silesian Belt
60: Shoulder Strap

What is claimed is:

1. An exoskeleton device, comprising:
a telescoping rod comprising at least one lower telescoping section and at least one an upper telescoping section;
a fracture stabilization orthosis;
at least one fracture suspension mechanism connecting the at least one lower telescoping section to the fracture stabilization orthosis; and
a thigh corset connectable to the at least one upper telescoping section.

2. The exoskeleton device according to claim 1, wherein the at least one lower telescoping section and at least one upper telescoping section are in slidable engagement.

3. The exoskeleton device according to claim 1, wherein the telescoping rod has an elliptical cross section or a pin on the stationary pylon and slot on moving pylon to prevent rotation.

4. The exoskeleton device according to claim 1, further comprising a knee joint connectable to the at least one lower telescoping section and at least one upper telescoping section, wherein the knee joint has a double hinge structure that allows the exoskeleton device to be folded/unfolded and locked on either a right side or a left side of a user's body.

5. The exoskeleton device according to claim 4, wherein the knee joint has a spring-loaded latch mechanism to release or lock a hinge structure.

6. The exoskeleton device according to claim 1, further comprising an end effector connectable to an end of the at least one lower telescoping section to provide the device with a foot-like structure, wherein the end effector is foldable up to and/or at least partially around the telescoping rod.

7. The exoskeleton device according to claim 1, wherein the at least one fracture suspension mechanism comprises a linkage structure comprising at least one crossbar extending from the at least one lower telescoping section to the fracture stabilization orthosis.

8. The exoskeleton device according to claim 7, wherein the linkage structure comprises two to four crossbars.

9. The exoskeleton device according to claim 1, comprising two fracture suspension mechanisms, each comprising a body having a cavity that is in free and axial slidable engagement with the at least one lower telescoping section and a concave surface that is positioned against the fracture stabilization orthosis.

10. The exoskeleton device according to claim 1, wherein the fracture stabilization orthosis comprises a fabric material wrappable around a lower leg and has a frustrum shape.

11. The exoskeleton device according to claim 1, wherein the fracture stabilization orthosis comprises a fabric wrap having a plurality of pockets, with a strut insertable into each pocket.

12. The exoskeleton device according to claim 11, wherein the fracture stabilization orthosis comprises two to twelve struts, each strut comprising at least one sheet of ultra-high-molecular-weight polyethylene, at least one sheet of aluminum, at least one sheet of a unidirectionally woven carbon fiber or fiberglass, or any combination thereof.

13. The exoskeleton device according to claim 1, wherein the thigh corset comprises a fabric material wrappable around a user's thigh.

14. The exoskeleton device according to claim 13, wherein the thigh corset comprises a plurality of pockets, each pocket for receiving one or more stays.

15. The exoskeleton device according to claim 1, further comprising an ischial sling connectable to the at least one upper telescoping section above the thigh corset and wrappable underneath a user's pelvis.

16. The exoskeleton device according to claim 15, wherein the ischial sling comprises a strip of fabric extending or threaded through a foam tube serving as a pad.

17. The exoskeleton device according to claim 15, further comprising a silesian belt, wherein the ischial sling and silesian belt comprise a single strip of material.

18. The exoskeleton device according to claim 1, further comprising a shoulder strap.

19. The exoskeleton device according to claim 18, wherein the shoulder strap, fracture stabilization orthosis, thigh corset, an ischial sling, each have a buckle which is not interoperable with another buckle.
